(19) United States
(12) Patent Application Publication
(10) Pub. No.: US 2010/0145165 A1
(43) Pub. Date: Jun. 10, 2010

(54) Notification System

(75) Inventor: Alan Forbes Merry, Auckland (NZ)

Correspondence Address: WADDEY & PATTERSON, P.C., 1600 DIVISION STREET, SUITE 500, NASHVILLE, TN, 37203, US

(73) Assignee: Safer Sleep Limited

(21) Appl. No.: 12/521,661

(22) PCT Filed: Dec. 20, 2007
(86) PCT No.: PCT/NZ07/00385

§ 371 Date: Jan. 20, 2010

(30) Foreign Application Priority Data
Dec. 30, 2006 (NZ) 548290

(51) Int. Cl. A61B 5/08; G06Q 50/00; A61B 5/00

(52) U.S. Cl. 600/301; 705/2; 600/300; 600/532

(57) ABSTRACT

The invention relates to a process and apparatus for notifying medical staff that an event or action is due on the basis of one or more physiological variation of a patient. In particular the invention relates to a notification system for anaesthetists to provide medication.

### FIELD OF THE INVENTION

The present invention relates to a process and apparatus for notifying medical staff that an event or action is due on the basis of one or more physiological variables of a patient. The invention also provides a means for reducing the likelihood of post-operative infection.

### BACKGROUND

A variety of physiological variables are recorded when anesthetising a patient prior to a surgical process. Typically the end tidal CO₂ gas concentration levels exhaled from a patient are recorded, along with end tidal O₂ concentration, pulse rate, brain waves and the like.

This data may be useful in developing a profile of the patient to assist the anaesthetist and ensure safety to the patient. Vital life signs are monitored to ensure no adverse effects are caused by administration of certain sedatives and anaesthetic drugs. The data also allows the anaesthetist to determine suitable times for administration of additional drugs, other anaesthetics or when to commence surgery.

Much of this data is displayed in a raw form on visual display units (VDU) or on print-outs. Currently the anaesthetist must review this information and form a judgement on whether the patient is in a suitably anesthetised state to administer additional drugs and/or whether the surgery should proceed.

Typically much activity occurs in an operating theatre and an anaesthetist can be temporarily distracted by surrounding events. This may adversely impart on the best time to administer a drug. In addition, the assessment of the data is completely dependent on the anaesthetist's skill and knowledge.

### OBJECT OF THE INVENTION

It is an object of the present invention to provide a process and apparatus for notifying medical staff that an event or action is due on the basis of one or more physical variables. It is a further or alternative object of the invention to at least provide the public with a useful choice.

### SUMMARY OF THE INVENTION

According to a first aspect of the invention there is provided an apparatus for notifying medical staff that an event or action is due, the apparatus including:
a processor adapted to receive variable physiological data from a patient and determine whether the data has exceeded one or more predetermined threshold values; and
a display associated with the processor and adapted to indicate that the predetermined threshold value(s) has been exceeded a predetermined number of times during an interval, thereby prompting the medical staff.

Preferably the processor is adapted to determine when an antibiotic should be administered in an anaesthetic procedure.

Preferably the variable physiological data is recorded by a sensor.

Preferably the sensor feeds the variable physiological data continuously to the processor.

Preferably the sensor is located within a ventilating unit and is adapted to record the concentration of constituents of gas exhaled by the patient.

Preferably the display is an audio and/or visual display unit (VDU).

Preferably the event or action includes the administration of an antibiotic.

Preferably the predetermined threshold value is set at about 3 mm_{of}Hg or greater when the sensor records tidal CO₂ concentration exhaled from a patient.

Preferably the predetermined threshold value is selected from between 3 to 10 mm_{of}Hg when the sensor records end tidal CO₂ concentration exhaled from the patient.

Preferably the number of times the predetermined threshold value is exceeded is set at 2 or more before triggering a display indication.

Preferably the display is adapted to indicate that the predetermined threshold value has been exceeded a predetermined number of times by a dialogue box appearing on a VDU optically with a sound indicator.

Preferably the interval is 10 seconds to 1 minute.

Preferably, the apparatus is adapted to receive and/or record and/or display additional data.

According to a further aspect of this invention there is provided a process for notifying medical staff that an event or action is due, the process including the steps of:
recording a variable physiological value from a patient's on the basis of their physiology;
determining whether the variable physiological value exceeds one or more predetermined threshold values; and
indicating by audio and/or visual means whether the predetermined threshold value(s) has been exceeded a predetermined number of times during an interval, thereby prompting medical staff.

Preferably the event or action includes administering an antibiotic.

Preferably a sensor records the patient's physiology.

Preferably the sensor is adapted to record end tidal CO₂ concentration, end tidal concentration of anaesthetic vapour or gas, pulse rate, electrocardiograph (ECG), blood pressure, end tidal O₂ concentration, or bispectral index (BIS).

Preferably the interval is 1 minute.

Preferably the predetermined threshold value is 3 mm_{of}Hg or greater when the sensor records tidal CO₂ concentration exhaled from a patient.

Preferably the predetermined threshold value is 3 to 10 mm_{of}Hg when the sensor records end tidal CO₂ concentration exhaled from a patient.

According to a further aspect of this invention there is provided a method of improving patient safety during surgery by providing a visual and/or audio display sign triggered by variable physiological data recorded from the patient.

According to a further aspect of this invention there is provided a method of reducing post-operative infection including the step of administering

According to a further aspect of the invention there is provided an apparatus for notifying medical staff that an event or action is due substantial as herein described with reference to the attached Example.

According to a further aspect of this invention there is provided a process for notifying medical staff that an event or action is due substantially as herein described with reference to the attached Example.

According to a further aspect of this invention there is provided a method of improving patient safety during surgery substantially as herein described with reference to the attached Example.

### PREFERRED EMBODIMENT OF THE INVENTION

The present invention relates to an apparatus and process for notifying medical staff, and more particularly anaesthetists, that an event or action is due, or required. The event or action may include the administering of an antibiotic during an anaesthetic process. Administering an antibiotic during the anaesthetic process has been found to reduce post-operative infection.

The present invention is described with reference to administering an antibiotic to a subject or patient, although it will be appreciated that the invention is not restricted to administration of antibiotics or drugs alone. The invention may be used to indicate when it is appropriate for surgery to be commenced, or the administration of further anaesthetic for example. The invention has particularly been developed with patient safety in mind by notifying an anaesthetist of the patient's current anaesthetised state.

The processor unit or controller is generally in the form of a console unit. The processor is adapted to receive and process electronic data recorded from at least one sensor associated with the subject or patient. The processor unit may further receive and/or record additional data such as drugs already administered and the like.

The sensor(s) records a patient's variable physiological condition. The sensor(s) selected may detect and/or record a variety of variable biological or physiological data including the end tidal CO₂ concentration, the end tidal concentration of anaesthetic vapour or gas, pulse rate, electrocardiogram (ECG) data, blood pressure, end tidal CO₂ concentration, or bispectral index data.

Sensors which record and analyse such data are known in the art. If the sensor records tidal CO₂, O₂ or anaesthetic vapour concentrations exhaled from the patient typically the data originates from a sensor encased within a ventilation unit, which breaths for the patient following the administration of an induction agent and muscle relaxant. In addition, a variety of sensors connected to the patient may record data and feed such data to the processor. Such sensors physically connected to the patient can record pulse rate, blood pressure and the like.

Commonly known sensors may be suitable to feed data to the processor of the present invention. However particular sensors may be developed to improve the accuracy and assist in comparing the recorded variable physiological data with a predetermined threshold value.

The sensor(s) may continuously monitor the subject's variable physiological condition and transfer the data to the processor. The processor may be adapted to monitor and record the data transferred from the sensor(s) and may store and display additional data, such as drugs already administered and the like.

The processor is also adapted to determining whether the data has increased or decreased beyond one or more predetermined threshold value.

The threshold value(s) may be programmed into the processor before variable physiological data is obtained. The particular threshold value selected will depend on what data is to be analysed and in particular, the type of physiological data the sensor records.

For example, if the processor is adapted to display the optimum time for administering an antibiotic, on the basis of a tidal CO₂ concentration sensor, preferably the threshold value will be selected from a value between 3 mm_{of}Hg to 10 mm_{of}Hg. This threshold value range is coincident with a subject under the influence of anaesthetic, whereby a ventilator unit “breaths” for the subject.

The present invention also includes a signalling means for indicating or notifying a user that an action or event is required or due. By way of example, a required event may be signalled by way of a display. Typically, the display is associated and/or connected with the processor. The display may include a visual and/or audio means.

The display is typically a visual display unit (VDU) having sound capabilities. Preferably the VDU displays the data detected by the sensors. The display indicates when one or more predetermined threshold values have been exceeded a predetermined number of times during an interval, thereby prompting the medical staff to act (e.g. administer an antibiotic).

In the preferred embodiment, a dialogue box is displayed by the VDU when the threshold value has been exceeded a predetermined number of times during a defined interval. The dialogue box may state a phrase for example: “Warning, antibiotic yet to be administered”. Preferably the dialogue box requires the anaesthetist to take action.

In the preferred embodiment, the VDU indicates when the predetermined threshold value has been exceeded two or more times by the variable physiological data over an interval of 10 seconds to 1 minute.

It will be appreciated that the number of times the threshold is exceeded and the duration of the interval may all be varied depending on when an event is required to be triggered and on the basis of what physiological data the sensor records.

The signalling means may further indicate additional data received from the processor, such as drugs that have been given or not give.

It will be further appreciated that the predetermined parameters, such as the threshold values or the number of times the threshold values may be exceeded, may be selected in accordance with the action or event being notified. Furthermore, the patient's variable physiological condition may be assessed by determining one or more of a variety of the patient's physiological signs, such as end tidal CO₂ concentration, end tidal concentration of anaesthetic vapour or gas, pulse rate, electrocardiograph (ECG), blood pressure, end tidal O₂ concentration, or bispectral index (BIS). These signs may be used in singularity or in combination to determine when an action or event is required or due. For example, these signs may be used to continuously monitor a patients anaesthetic status to determine events such as an appropriate time for the commencement of surgery.

The present invention also provides a process for notifying medical staff that an event is due, for example, notifying medical staff that an antibiotic should be administered.

The preferred process will be described below with respect to a sensor recording a tidal CO₂ concentration exhaled from a patient. The predetermined threshold value has been set relative to the method of detection and with respect to the optimum time to administer an antibiotic.

The process will be described below with reference to a typical anaesthetic process for anaesthetising a patient prior to surgery.

Initially a patient is prepared for surgery by bringing the patient into theatre. Either before the patient enters into the theatre or once located in the theatre, the patient receives a mild sedative. Appropriate sedatives are known in the art. The sedative has the effect of relaxing the patient physiologically, and particularly psychologically, for surgery.

A face mask is then affixed over the patient's mouth and nose. A high concentration of oxygen is supplied through the face mask onto the patient's face to ensure maximum oxygenation of tissue, particularly brain tissue, before the anaesthetic process. The patient is thereby in the beet possible state for receiving the anaesthetic.

An induction agent and muscle relaxant is then administered (generally) intravenously to the patient. Appropriate induction agent and muscle relaxant are known in the art.

At this point the muscles of the chest begin to fail as the muscle relaxant takes effect and the patient requires assisted respiration, initially by a hand held ventilator, and then from an automated ventilation unit. A tube is inserted down the windpipe of the patient and the tube is coupled to the ventilation unit which supplies air to the patient. The ventilation unit also receives the exhaled air from the patient.

Sensors within the ventilator unit may be provided in the downstream airflow of exhaled air to continuously measure end tidal CO₂ concentration, end tidal O₂ concentration, or end tidal concentrations of anaesthetic vapour or gas, for example. Date recovered from these sensors may be feed in an electronic form into the apparatus of the present invention.

As mentioned above, the invention includes a processor adapted to receive variable physiological data from a patient and determine whether the data has exceeded one or more predetermined threshold values.

If the variable physiological data relied upon originates from a sensor recording end tidal CO₂ concentration, the predetermined threshold value(s) may be set between 3 mm_{of}Hg to 10 mm_{of}Hg, and more preferably 3 mm_{of}Hg.

In one preferred embodiment, the processor scans for two variable physiological values which exceed the predetermined threshold value during an interval of about 1 minute. Of course, the processor may be set to record one, two, three of more values exceeding the predetermined threshold value depending on the type of sensor and the particular event or action to be signalled by the apparatus.

In the preferred embodiment, once two values are recorded above the predetermined threshold value, a dialogue box appears on the VDU. As mentioned above, the VDU may display a warning such as “Warning, antibiotic yet to be administered”. Of course the dialogue box warning could recite any particular phrase depending on what was required by the user. Furthermore the dialogue box warning could be personalised to a particular user.

The software may be adapted so that once a dialogue box appears, the medical staff member (e.g. generally anaesthetist) is required to actively respond to the processor by acknowledging the box and thereby confirming that the antibiotic is or has been administered to the patient. Alternatively, the box may be postponed for a period of time. The invention has the advantage of at least prompting the anaesthetist to cast his or her mind towards the issue of administering a drug or taking some action. The present invention could be used to indicate a suitable time to commence surgery, for example.

As mentioned, the above example relates specifically to the use of end tidal CO₂ concentration in a patient's exhaled air to trigger an action by a medical staff member through visual or audio means. The processor may be adapted to process information in the same or similar way as described where the sensor records brain waves, end tidal concentration of anaesthetic vapour or gas, pulse rate, electrocardiogram (ECG) data, blood pressure, or bispectral index data, for example. In these cases, the predetermined threshold value may be varied appropriately.

The present invention therefore provides an effective means of prompting medical staff, and particularly an anaesthetist, that a certain event or action is required. The invention has a variety of applications including, but not limited to:
prompting administration of drugs to a patient;
indicating the appropriate time for surgery to commence; and
indicating when a patient may require a further anaesthetic to ensure a patient remains in an unconscious state during surgery.

The invention also provides a safety element in that it provides a confirmatory means to ensure that a correct and appropriate drug is administered to a patient during a surgical process at an optimum time based on their physiological state. The invention can also provide a means of tracking what has been administered to a patient during the course of surgery.

The invention also provides a method of improving patient safety during surgery by providing a visual and/or audio display sign triggered by variable physiological data recorded from the patient.

The invention is further described in Example 1 which has been excised from a manual and is by example only.

### Example 1

This document is a functional specification detailing the Antibiotic warning functionality of SAFERsleep 4.9.0 system and how it can be implemented.

1 Overview

Antibiotic prophylaxis has been shown to significantly reduce post-operative infection. Version 4 of the SAFERsleep OR client version does not currently remind anaesthetists to administer antibiotics. This reminder will be added as part of release 4.9.0.
The antibiotic warning functionality will remind the anaesthetist to administer an antibiotic at the beginning of the anaesthetic.

Here is a brief summary of how this module works:

At the beginning of the anaesthetic case, a dialog is displayed reminding the anaesthetist to administer an antibiotic.

If the anaesthetist elects to be reminded again, the dialog will be displayed at a set time interval later in the case, until an antibiotic drug is administered or the user states that an antibiotic will not be required for this case.

If an antibiotic drug is administered, the antibiotic warning dialog will not appear when the timer initiates another check. An entry will be posted in the internal log. The antibiotic warning will not appear again in case the antibiotic drug is marked as being not administered and no other antibiotic drugs have been administered.

2 Major Features

2.1 Display the First Reminder when Anaesthetic Starts

The reminder dialog will be displayed when the anaesthetic procedure starts. The definition of the start of a procedure is defined later in this document.

2.2 Display a Second Reminder at Periodic Intervals

Once the first dialog has been displayed, a timer will be started. When the timer fires, another reminder will be displayed. This reminder will continue to be displayed at the set interval until either the anaesthetist acknowledges the reminder or an antibiotic is administered.

2.3 Disable Antibiotic Warnings for the Client

Although enabled by default, settings in the client INI file will allow the antibiotic warning to be disabled.

2.4 Diagnostic Logging

A new logging system will be included in release 4.9. This will be used to record key user actions and will be stored in the server database along with the anaesthetic.

Each diagnostic log entry will contain:
1. A timestamp
2. The Windows PC name (e.g. ssl-filesvr)
3. The machine number as defined in IDAS.ini
4. The logged in user name (or guest)
5. A separator |
6. The application area the log event is from (in brackets [ ])
7. Specific information about the logged event

e.g. 29/12/2005 12:12:52 TESTER 1 ahenderson | [AntibioticWarning] Form displayed

In the case of antibiotic warning, the application area will be “AntibioticWarning” and the specific information will record which button the user pressed, or if the dialog was closed by the system (for example by a barcode scan presenting a new dialog)

If an antibiotic has been administered, an entry is posted in the internal log. e.g. 29/12/2005 12:17:53 TESTER 1 ahenderson | [AntibioticWarning] An antibiotic has been administered. This entry will not contain the name of the antibiotic that was given.

3 Typical Scenarios

### I:

An anaesthetist starts a new case, and forgets to administer an antibiotic. When the first series of CO2 readings are recorded, a dialog is displayed reminding them to administer an antibiotic.

The anaesthetist clicks the dialog's “acknowledged” button, but does not administer the antibiotic. The reminder dialog is displayed later.

### II:

An anaesthetist starts a new case. The first drug administered, before the first valid CO2 readings are received, is an antibiotic. They never see the reminder message.

### III:

An anaesthetist starts a new case and before they administer the first drug, the first non-zero CO2 reading series is returned from the anaesthetic monitor. This causes the dialog to be displayed. The anaesthetist acknowledges the reminder and administers an antibiotic. The reminder is never displayed again.

### IV:

An anaesthetist starts a new case, grabs two drugs and scans them quickly one after the other. None of these drugs are antibiotics. At the same time, the first non-zero CO2 reading series is received.

The warning dialog is displayed, but is then closed by a drug scan before the anaesthetist can acknowledge it. The audio warning is heard. In this case, the dialog will be closed as if the “acknowledge” button was pressed, so the warning dialog will be displayed later.

### V:

An anaesthetist starts the application and then starts a new case. The first CO2 reading series is received. The anaesthetist does not intend giving any antibiotics for this list so she clicks the “Not Required” button. No further antibiotic warnings are displayed.

4 Software Architecture

Most of the GUI logic is written in C++ Builder. Delphi is used for a small number of GUI forms and web service interfaces.

Because the bulk of the client is written in C++, this will be the language used to implement the antibiotic warning functionality.

5 Design Considerations

5.1 First Reminder

The trigger that causes the reminder dialog to be displayed is the start of a case.

Determining the start of a case is not straightforward. It is not when “new anaesthetic” is selected from the menu. For the purposes of this module, the start of a case will be determined to be the first series of valid CO2 readings.

A series of valid readings is all readings over a set time interval that are above the designated minimum value. We cannot check for zero values, since we may receive low-value “ghost” readings.

The process for measuring the first series of valid CO2 readings is as follows:

5.2 Subsequent Reminders

If the user selects the “Acknowledge” button when the reminder dialog is displayed, the dialog will be closed. After an interval specified in configuration, another check will be made to see if an antibiotic has been administered. If no, the dialog will be displayed again. When “Acknowledge” is pressed, a timer will be created, to wait the appropriate period of time before displaying the dialog again.

5.3 Class Model

Note that when a case is retrieved from a park queue, a check is made to see if an antibiotic has been given. If not, the warning feature is reset, irrespective of any administered drugs or readings. This covers the situation where the case is started and the patient prepped in one room by an anaesthetist and a different anaesthetist continues the case once it reaches the OR.

While it would be possible to have a background thread checking for these, a simpler solution would be to use events. An event delegate will be implemented that will fire when a CO2 reading is received.

When the first valid reading is received, a timer will be started and set to run once, setting the interval to the sample Interval configuration value. A check will be made when the timer's tick event fires, to determine if the case can be considered started.

It is assumed that other functionality could reuse the new event delegates. For example, a CO2 alert could check CO2 readings and display a warning when CO2 values approached zero.

6 GUI Overview

The only GUI component of this feature is the alert message. It is assumed that this will be a modal dialog—it will remain as the front-most screen in the application until it is closed.

6.1 Dialog Text

The functional specification may have the following message and buttons:
Dialog message: “Reminder: Antibiotic not yet administered”
Button: “Not Required”
Button: “Acknowledged”

When the dialog is displayed, audio will be played: “Antibiotic not yet administered”.

If the user clicks “Not Required”, no further antibiotic warnings will be displayed for this anaesthetic.

If the user clicks “Acknowledged”, the dialog will be displayed at a later interval, unless an antibiotic is administered in the interim.

Implementation note: Be sure the ShowPopupAndContinue( ) method is used instead of the standard ShowModal( ). This to ensure that the remainder of the system will continue to process barcode scans.

If the user scans a drug while the antibiotic alert is being displayed, the alert will be closed automatically and the drug scan dialog will be displayed as normal. This is the default action for dialogs throughout the system. This entry in the internal log is identical to the anaesthetist clicking on the “Acknowledged” button. It can not be determined whether the antibiotic warning dialog was automatically closed by a barcode scan or by human intervention.

This could result in the alert dialog being displayed and then closed rapidly by the subsequent drug scan.

7 Configuration and State

The different criteria for determining the start of a case can be enabled or disabled in the configuration file.

To avoid hard-coding, the name of the antibiotic drug class name will be stored in the configuration file.

A configuration setting may be placed into the configuration file to disable the Antibiotic warning feature if necessary. If the feature is disabled here then this setting will override any other setting for it.

When the first antibiotic warning dialog is displayed at the start of a case the user will be able to turn the antibiotic warning feature off. When the application is restarted the feature will be turned on by default.

Configuration settings required:

| Setting | Contains |
|---|---|
| antibioticwarning.enable.warning.on.co2.reading | If true, the warning on the first CO2 |
| | readings will be enabled. |
| antibioticwarning.co2.reading.minim.value | The minimum CO2 reading value that will |
| | be considered a valid reading. |
| antibioticwarning.co2reading.sampleinterval | The amount of time between receiving the |
| | first valid CO2 reading and counting the |
| | total number received. |
| antibioticwarning.co2.reading.minimum.sample.count | The minimum number of valid readings that |
| | must be received within the sample interval |
| | for the anaesthetic to be considered |
| | started. |
| antibioticwarning.acknowledge.reminder.interval | The amount of time (in seconds) between |
| | displaying the reminder, if the anaesthetist |
| | clicks the “acknowledge” button. |
| antibioticwarning.antibiotic.drug.class | The name of the antibiotic drug class. This |
| | will be used to determine if any antibiotics |
| | have been administered. |
| antibioticwarning.reminder.sound.file | The name of the sound file that will be |
| | played when the antibiotic warning is |
| | displayed. |

Where in the foregoing description, reference has been made to specific components or integers of the invention having known equivalents then such equivalents are herein incorporated as if individually set forth.

Although this invention has been described by way of example and with reference to possible embodiments thereof, it is to be understood that modifications or improvements may be made thereto without departing from the scope or spirit of the invention.

1-32. (canceled)

33. A notification apparatus comprising:
a processor adapted to receive variable physiological data from a subject and determine whether the data has increased or decreased beyond one or more predetermined threshold value(s) a predetermined number of times during a predetermined interval; and
a signaling means adapted to indicate at least one event or action is required, including the administration of a drug.

34. A notification apparatus according to claim 33, wherein the drug is an antibiotic, heparin or protamine.

35. A notification apparatus according to claim 34, wherein the variable physiological data is indicative of a patient's anaesthetized state.

36. A notification apparatus according to claim 33, wherein the physiological data is determined by at least one sensor.

37. A notification apparatus according to claim 36, wherein the sensor(s) is located within a ventilating unit and is adapted to continuously determine the concentration of gas constituents exhaled by a subject.

38. A notification apparatus according to claim 36, wherein the sensor(s) is adapted to determine the tidal CO₂ concentration exhaled by the subject.

39. A notification apparatus according to claim 33, wherein the predetermined threshold value is set at about 3 mm_{of}Hg or greater when the sensor(s) records the tidal CO₂ concentration exhaled by a human subject.

40. A notification apparatus according to claim 33, wherein the signaling means indicates an event or action is due by way of visual and/or audio signal.

41. A notification apparatus according to claim 40, wherein the signal is displayed at predetermined intervals until the signal is acknowledged and/or an antibiotic is administered.

42. A notification apparatus according to claim 33, wherein the apparatus includes a confirmatory means to ensure that a correct and appropriate drug is administered to a subject at an optimum time based on the physiological data.

43. A notification apparatus according to claim 33, wherein a required action or event includes the commencement of surgery.

44. A notification apparatus according to claim 33, wherein a required action or event includes the administration of further anesthetic.

45. A notification process including the steps of:
determination of variable physiological and other data for a subject,
determination of whether the physiological data increases or decreases beyond a predetermined threshold value, and
indicating at least one event or action is required, including the administration of a drug, when the predetermined threshold value(s) has been passed a predetermined number of times during a predetermined interval.

46. A notification process according to claim 45, wherein the drug is an antibiotic, heparin or protamine.

47. A notification process according to claim 45, wherein the variable physiological data is indicative of a patient's anaesthetized state.

48. A notification process according to claim 45, wherein at least one sensor(s) determines the variable physiological data from a subject.

49. A notification process according to claim 48, wherein the sensor(s) is adapted to determine end tidal CO₂ concentration, end tidal concentration of anesthetic vapor or gas, pulse rate, electrocardiograph (ECG), blood pressure, end tidal O₂ concentration and/or bispectral index (BIS).

50. A process according to claim 45, wherein the predetermined threshold value is 3 mm_{of}Hg or greater when the sensor records tidal CO₂ concentration exhaled from a human subject.

51. A method of improving patient safety during surgery by providing a visual and/or audio display sign indicating at least one event or action is required, including the administration of a drug, the sign being activated by variable physiological data recorded from the patient.

52. A method of improving patient safety during surgery as claimed in claim 51, wherein the drug is an antibiotic, heparin or protamine.
